SEQUENCE LISTING (8 sequences)

SEQ ID NO: 1 - protein, 1173 aa, synthetic construct
  REGION 1..1173: note = rVAR2-1
SDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDGNDVTFFNLFEQWNKEIQYQIEQYMTNANISCIDEKEVLDSVSDEGTPKVRGGYEDGRNNNTDQGTNCKEKCKCYKLWIEKINDQWGKQKDNYNKFRSKQIYDANKGSQNKKVVSLSNFLFFSCWEEYIQKYFNGDWSKIKNIGSDTFEFLIKKCGNNSAHGEEIFSEKLKNAEKKCKENESTDTNINKSETSCDLNATNYIRGCQ

SEQ ID NO: 2 - protein, 589 aa, synthetic construct
  REGION 1..589: note = rVAR2-2
QAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLD

SEQ ID NO: 3 - protein, 581 aa, synthetic construct
  REGION 1..581: note = rVAR2-3
QEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLD

SEQ ID NO: 4 - protein, 795 aa, synthetic construct
  REGION 1..795: note = rVAR2-4
QAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDGNDVTFFNLFEQWNKEIQYQIEQYMTNANISCIDEKEVLDSVSDEGTPKVRGGYEDGRNNNTDQGTNCKEKCKCYKLWIEKINDQWGKQKDNYNKFRSKQIYDANKGSQNKKVVSLSNFLFFSCWEEYIQKYFNGDWSKIKNIGSDTFEFLIKKCGNNSAHGEEIFSEKLKNAEKKCKENESTDTNINKSETSCDLNATNYIRGCQ

SEQ ID NO: 5 - DNA, 4731 bp, synthetic construct
  source 1..4731: mol_type = other DNA
  misc_feature 1..4731: note = rVAR2-1
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccggcggtggcggtggctcgggcggaggtgggtcgggtggcggcggatcaggtctggaagttctgttccaggggcccggtgaattcagcgacagcggcaagtacgatccgtgcgagaagaaactgccgccgtatgacgataacgatcagtggaagtgccagcaaaacagcagcgacggtagcggcaaaccggaaaacatctgcgttccgccgcgtcgtgagcgtctgtgcacctacaacctggaaaacctgaagttcgacaaaattcgtgataacaacgcgtttctggcggatgtgctgctgaccgcgcgtaacgagggcgaaaagatcgttcaaaaccacccggacaccaacagcagcaacgtgtgcaacgcgctggagcgtagctttgcggacctggcggatatcattcgtggtaccgaccagtggaaaggcaccaacagcaacctggagaagaacctgaaacaaatgtttgcgaagatccgtgaaaacgacaaagttctgcaggataagtacccgaaagaccaaaagtataccaaactgcgtgaggcgtggtggaacgcgaaccgtcagaaagtttgggaagtgattacctgcggtgcgcgtagcaacgatctgctgatcaagcgtggttggcgtaccagcggcaaaagcgaccgtaagaaaaacttcgagctgtgccgtaagtgcggccactacgagaaagaagttccgaccaagctggattatgtgccgcagttcctgcgttggctgaccgagtggattgaagacttttaccgtgagaaacaaaacctgatcgacgatatggaacgtcaccgtgaggaatgcacccgtgaggatcacaagagcaaagaaggtaccagctactgcagcacctgcaaggacaaatgcaagaaatattgcgaatgcgttaagaaatggaagaccgagtgggaaaaccaggagaacaagtataaagacctgtacgaacagaacaagaacaaaaccagccagaagaacaccagccgttatgacgattacgtgaaagatttctttgagaagctggaggcgaactacagcagcctggagaactatattaagggtgacccgtatttcgcggaatacgcgaccaaactgagctttatcctgaacccgagcgatgcgaacaacccgagcggcgagaccgcgaaccacaacgacgaggcgtgcaactgcaacgaaagcggtattagcagcgtgggtcaggcgcaaaccagcggtccgagcagcaacaagacctgcattacccacagcagcatcaaaaccaacaagaaaaaggagtgcaaggatgttaaactgggcgtgcgtgaaaacgacaaggatctgaaaatctgcgttattgaggacaccagcctgagcggtgtggataactgctgctgccaggacctgctgggcatcctgcaagaaaactgcagcgataacaagcgtggtagcagcagcaacgacagctgcgataacaaaaaccaggacgagtgccagaagaagctggaaaaggttttcgcgagcctgaccaacggttacaaatgcgataagtgcaaaagcggcaccagccgtagcaaaaagaaatggatttggaagaaaagcagcggtaacgaggaaggcctgcaggaagagtacgcgaacaccatcggtctgccgccgcgtacccaaagcctgtatctgggcaacctgccgaagctggagaacgtttgcgaagacgtgaaagatattaacttcgacaccaaggagaaatttctggcgggttgcctgatcgtgagcttccacgaaggcaagaacctgaagaaacgttacccgcagaacaaaaacagcggtaacaaggagaacctgtgcaaagcgctggaatatagctttgcggactacggtgatctgattaagggcaccagcatctgggataacgagtataccaaagacctggaactgaacctgcagaacaacttcggcaagctgttcggcaagtacattaagaaaaacaacaccgcggagcaagataccagctatagcagcctggacgagctgcgtgaatcctggtggaacaccaacaagaaatacatttggaccgcgatgaaacacggtgcggaaatgaacatcaccacctgcaacgcggatggcagcgttaccggtagcggcagcagctgcgacgatatcccgaccattgacctgatcccgcagtatctgcgtttcctgcaagagtgggtggaaaacttttgcgagcagcgtcaagcgaaggttaaagatgtgatcaccaactgcaagagctgcaaagagagcggtaacaagtgcaaaaccgaatgcaagaccaaatgcaaggacgagtgcgaaaagtacaagaaatttatcgaggcgtgcggtaccgcgggtggcggtattggtaccgcgggtagcccgtggagcaaacgttgggatcagatttataagcgttacagcaaacacatcgaggacgcgaagcgtaaccgtaaagcgggtaccaagaactgcggtaccagcagcaccaccaacgcggcggcgagcaccgatgaaaacaagtgcgttcaaagcgacattgatagcttctttaaacacctgatcgacattggtctgaccaccccgagcagctacctgagcaacgtgctggacgataacatttgcggtgcggacaaggcgccgtggaccacctataccacctacaccaccaccgagaagtgcaacaaagaacgtgataaaagcaagagccagagcagcgacaccctggtggttgtgaacgttccgagcccgctgggtaacaccccgtaccgttataagtacgcgtgccagtgcaaaatcccgaccaacgaggaaacctgcgacgatcgtaaagagtacatgaaccaatggagctgcggtagcgcgcgtaccatgaaacgtggctataagaacgataactacgaactgtgcaagtataacggtgttgacgtgaaaccgaccaccgttcgtagcaacagcagcaagctggacggcaacgatgtgaccttctttaacctgttcgagcagtggaacaaagaaattcagtaccaaatcgagcaatatatgaccaacgcgaacattagctgcatcgatgagaaggaagttctggacagcgtgagcgatgagggtaccccgaaagtgcgtggcggttacgaagatggtcgtaacaacaacaccgaccagggcaccaactgcaaagagaagtgcaaatgctataagctgtggattgaaaaaatcaacgatcagtggggcaagcaaaaagacaactacaacaaatttcgtagcaagcagatttatgacgcgaacaagggcagccagaacaagaaagttgtgagcctgagcaacttcctgttctttagctgctgggaggaatacatccagaagtacttcaacggtgattggagcaagattaaaaacatcggcagcgacaccttcgaatttctgattaagaaatgcggtaacaacagcgcgcacggcgaggaaatcttcagcgagaagctgaaaaacgcggaaaagaaatgcaaggagaacgaaagcaccgataccaacattaataaaagcgagaccagctgcgacctgaacgcgaccaactacatccgtggttgccagaagcttggtggcaagccgatcccgaacccgctgctgggtctggacagcaccggtgaaaacctgtattttcaaggccaccatcatcaccatcatcaccaccaccacctcgagcggccgcatcgtgactgactgacgatctgcctcgcgcgtttcggtgatgacggtgaaaacctctgacacatgcagctcccgg

SEQ ID NO: 6 - DNA, 2979 bp, synthetic construct
  source 1..2979: mol_type = other DNA
  misc_feature 1..2979: note = rVAR2-2
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccggcggtggcggtggctcgggcggaggtgggtcgggtggcggcggatcaggtctggaagttctgttccaggggcccggtgaattccaggcgcaaaccagcggtccgagcagcaacaagacctgcattacccacagcagcatcaaaaccaacaagaaaaaggagtgcaaggatgttaaactgggcgtgcgtgaaaacgacaaggatctgaaaatctgcgttattgaggacaccagcctgagcggtgtggataactgctgctgccaggacctgctgggcatcctgcaagaaaactgcagcgataacaagcgtggtagcagcagcaacgacagctgcgataacaaaaaccaggacgagtgccagaagaagctggaaaaggttttcgcgagcctgaccaacggttacaaatgcgataagtgcaaaagcggcaccagccgtagcaaaaagaaatggatttggaagaaaagcagcggtaacgaggaaggcctgcaggaagagtacgcgaacaccatcggtctgccgccgcgtacccaaagcctgtatctgggcaacctgccgaagctggagaacgtttgcgaagacgtgaaagatattaacttcgacaccaaggagaaatttctggcgggttgcctgatcgtgagcttccacgaaggcaagaacctgaagaaacgttacccgcagaacaaaaacagcggtaacaaggagaacctgtgcaaagcgctggaatatagctttgcggactacggtgatctgattaagggcaccagcatctgggataacgagtataccaaagacctggaactgaacctgcagaacaacttcggcaagctgttcggcaagtacattaagaaaaacaacaccgcggagcaagataccagctatagcagcctggacgagctgcgtgaatcctggtggaacaccaacaagaaatacatttggaccgcgatgaaacacggtgcggaaatgaacatcaccacctgcaacgcggatggcagcgttaccggtagcggcagcagctgcgacgatatcccgaccattgacctgatcccgcagtatctgcgtttcctgcaagagtgggtggaaaacttttgcgagcagcgtcaagcgaaggttaaagatgtgatcaccaactgcaagagctgcaaagagagcggtaacaagtgcaaaaccgaatgcaagaccaaatgcaaggacgagtgcgaaaagtacaagaaatttatcgaggcgtgcggtaccgcgggtggcggtattggtaccgcgggtagcccgtggagcaaacgttgggatcagatttataagcgttacagcaaacacatcgaggacgcgaagcgtaaccgtaaagcgggtaccaagaactgcggtaccagcagcaccaccaacgcggcggcgagcaccgatgaaaacaagtgcgttcaaagcgacattgatagcttctttaaacacctgatcgacattggtctgaccaccccgagcagctacctgagcaacgtgctggacgataacatttgcggtgcggacaaggcgccgtggaccacctataccacctacaccaccaccgagaagtgcaacaaagaacgtgataaaagcaagagccagagcagcgacaccctggtggttgtgaacgttccgagcccgctgggtaacaccccgtaccgttataagtacgcgtgccagtgcaaaatcccgaccaacgaggaaacctgcgacgatcgtaaagagtacatgaaccaatggagctgcggtagcgcgcgtaccatgaaacgtggctataagaacgataactacgaactgtgcaagtataacggtgttgacgtgaaaccgaccaccgttcgtagcaacagcagcaagctggacaagcttggtggcaagccgatcccgaacccgctgctgggtctggacagcaccggtgaaaacctgtattttcaaggccaccatcatcaccatcatcaccaccaccacctcgagcggccgcatcgtgactgactgacgatctgcctcgcgcgtttcggtgatgacggtgaaaacctctgacacatgcagctcccgg

SEQ ID NO: 7 - DNA, 2955 bp, synthetic construct
  source 1..2955: mol_type = other DNA
  misc_feature 1..2955: note = rVAR2-3
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccggcggtggcggtggctcgggcggaggtgggtcgggtggcggcggatcaggtctggaagttctgttccaggggcccggtgaattccaggaacaaatcagcgacccgagcagcaacaaaacctgcattacccacagcagcatcaaggcgaacaagaaaaaggtttgcaagcacgtgaaactgggtgttcgtgagaacgacaaagatctgcgtgtttgcgtgattgaacacaccagcctgagcggcgtggagaactgctgctgccaggatttcctgcgtatcctgcaagaaaactgcagcgacaacaaaagcggtagcagcagcaacggcagctgcaacaacaagaaccaggaagcgtgcgaaaagaacctggagaaagttctggcgagcctgaccaactgctacaaatgcgataaatgcaagagcgaacaaagcaaaaagaacaacaagaactggatttggaaaaagagcagcggtaaagagggtggcctgcagaaggaatatgcgaacaccattggcctgccgccgcgtacccaaagcctgtgcctggtggtttgcctggacgagaaaggtaaaaagacccaggaactgaagaacatccgtaccaacagcgagctgctgaaagaatggatcattgcggcgttccacgaaggcaagaacctgaaaccgagccacgagaaaaagaacgacgataacggcaaaaagctgtgcaaggcgctggagtatagctttgcggactacggtgatctgattaaaggcaccagcatctgggataacgagtacaccaaggacctggaactgaacctgcagaagattttcggtaaactgttccgtaagtacatcaaaaagaacaacaccgcggaacaagataccagctatagcagcctggacgagctgcgtgaatcctggtggaacaccaacaaaaagtacatctggctggcgatgaagcacggtgcgggcatgaacagcaccacctgctgcggtgatggcagcgtgaccggtagcggtagcagctgcgacgatatcccgaccattgacctgatcccgcagtatctgcgtttcctgcaagagtgggttgaacacttttgcaaacagcgtcaagagaaagttaagccggtgattgaaaactgcaagagctgcaaagagagcggtggcacctgcaacggcgagtgcaagaccgaatgcaaaaacaagtgcgaagtgtacaaaaagttcatcgaggattgcaaaggtggcgacggtaccgcgggtagcagctgggttaagcgttgggatcagatttacaaacgttatagcaagtacatcgaggacgcgaaacgtaaccgtaaggcgggtaccaaaaactgcggtccgagcagcaccaccaacgcggcggaaaacaaatgcgtgcagagcgacattgatagcttctttaagcacctgatcgacattggtctgaccaccccgagcagctacctgagcattgttctggacgataacatctgcggcgcggataaagcgccgtggaccacctataccacctacaccaccaccgagaagtgcaacaaagaaaccgacaaaagcaagctgcagcaatgcaacaccgcggtggttgtgaacgttccgagcccgctgggtaacaccccgcacggctacaaatatgcgtgccagtgcaagatcccgaccaacgaggaaacctgcgacgatcgtaaggagtacatgaaccaatggagctgcggtagcgcgcgtaccatgaagcgtggctataaaaacgataactacgaactgtgcaaatataacggtgttgacgtgaagccgaccaccgtgcgtagcaacagcagcaagctggacaagcttggtggcaagccgatcccgaacccgctgctgggtctggacagcaccggtgaaaacctgtattttcaaggccaccatcatcaccatcatcaccaccaccacctcgagcggccgcatcgtgactgactgacgatctgcctcgcgcgtttcggtgatgacggtgaaaacctctgacacatgcagctcccgg

SEQ ID NO: 8 - DNA, 3597 bp, synthetic construct
  source 1..3597: mol_type = other DNA
  misc_feature 1..3597: note = rVAR2-4
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccggcggtggcggtggctcgggcggaggtgggtcgggtggcggcggatcaggtctggaagttctgttccaggggcccggtgaattccaggcgcaaaccagcggtccgagcagcaacaagacctgcattacccacagcagcatcaaaaccaacaagaaaaaggagtgcaaggatgttaaactgggcgtgcgtgaaaacgacaaggatctgaaaatctgcgttattgaggacaccagcctgagcggtgtggataactgctgctgccaggacctgctgggcatcctgcaagaaaactgcagcgataacaagcgtggtagcagcagcaacgacagctgcgataacaaaaaccaggacgagtgccagaagaagctggaaaaggttttcgcgagcctgaccaacggttacaaatgcgataagtgcaaaagcggcaccagccgtagcaaaaagaaatggatttggaagaaaagcagcggtaacgaggaaggcctgcaggaagagtacgcgaacaccatcggtctgccgccgcgtacccaaagcctgtatctgggcaacctgccgaagctggagaacgtttgcgaagacgtgaaagatattaacttcgacaccaaggagaaatttctggcgggttgcctgatcgtgagcttccacgaaggcaagaacctgaagaaacgttacccgcagaacaaaaacagcggtaacaaggagaacctgtgcaaagcgctggaatatagctttgcggactacggtgatctgattaagggcaccagcatctgggataacgagtataccaaagacctggaactgaacctgcagaacaacttcggcaagctgttcggcaagtacattaagaaaaacaacaccgcggagcaagataccagctatagcagcctggacgagctgcgtgaatcctggtggaacaccaacaagaaatacatttggaccgcgatgaaacacggtgcggaaatgaacatcaccacctgcaacgcggatggcagcgttaccggtagcggcagcagctgcgacgatatcccgaccattgacctgatcccgcagtatctgcgtttcctgcaagagtgggtggaaaacttttgcgagcagcgtcaagcgaaggttaaagatgtgatcaccaactgcaagagctgcaaagagagcggtaacaagtgcaaaaccgaatgcaagaccaaatgcaaggacgagtgcgaaaagtacaagaaatttatcgaggcgtgcggtaccgcgggtggcggtattggtaccgcgggtagcccgtggagcaaacgttgggatcagatttataagcgttacagcaaacacatcgaggacgcgaagcgtaaccgtaaagcgggtaccaagaactgcggtaccagcagcaccaccaacgcggcggcgagcaccgatgaaaacaagtgcgttcaaagcgacattgatagcttctttaaacacctgatcgacattggtctgaccaccccgagcagctacctgagcaacgtgctggacgataacatttgcggtgcggacaaggcgccgtggaccacctataccacctacaccaccaccgagaagtgcaacaaagaacgtgataaaagcaagagccagagcagcgacaccctggtggttgtgaacgttccgagcccgctgggtaacaccccgtaccgttataagtacgcgtgccagtgcaaaatcccgaccaacgaggaaacctgcgacgatcgtaaagagtacatgaaccaatggagctgcggtagcgcgcgtaccatgaaacgtggctataagaacgataactacgaactgtgcaagtataacggtgttgacgtgaaaccgaccaccgttcgtagcaacagcagcaagctggacggcaacgatgtgaccttctttaacctgttcgagcagtggaacaaagaaattcagtaccaaatcgagcaatatatgaccaacgcgaacattagctgcatcgatgagaaggaagttctggacagcgtgagcgatgagggtaccccgaaagtgcgtggcggttacgaagatggtcgtaacaacaacaccgaccagggcaccaactgcaaagagaagtgcaaatgctataagctgtggattgaaaaaatcaacgatcagtggggcaagcaaaaagacaactacaacaaatttcgtagcaagcagatttatgacgcgaacaagggcagccagaacaagaaagttgtgagcctgagcaacttcctgttctttagctgctgggaggaatacatccagaagtacttcaacggtgattggagcaagattaaaaacatcggcagcgacaccttcgaatttctgattaagaaatgcggtaacaacagcgcgcacggcgaggaaatcttcagcgagaagctgaaaaacgcggaaaagaaatgcaaggagaacgaaagcaccgataccaacattaataaaagcgagaccagctgcgacctgaacgcgaccaactacatccgtggttgccagaagcttggtggcaagccgatcccgaacccgctgctgggtctggacagcaccggtgaaaacctgtattttcaaggccaccatcatcaccatcatcaccaccaccacctcgagcggccgcatcgtgactgactgacgatctgcctcgcgcgtttcggtgatgacggtgaaaacctctgacacatgcagctcccgg

(19) United States
(12) Patent Application Publication
(10) Pub. No.: US 2026/0200996 A1
(43) Pub. Date: Jul. 16, 2026

(54) RECOMBINANT VAR2CSA PROTEIN, AND PREPARATION METHOD AND USE THEREOF

(71) Applicant: SUN YAT-SEN UNIVERSITY CANCER CENTER (THE AFFILIATED CANCER HOSPITAL OF SUN YAT-SEN UNIVERSITY, THE, Guangzhou (CN)

(72) Inventors: Weihua Jia, Guangzhou (CN); Peifen Zhang, Guangzhou (CN); Ziyi Wu, Guangzhou (CN); Wenbin Zhang, Guangzhou (CN); Yongqiao He, Guangzhou (CN); Quankai Mu, Guangzhou (CN)

(21) Appl. No.: 19/136,813

(22) PCT Filed: Dec. 11, 2023
(86) PCT No.: PCT/CN2023/137976

§ 371 Date: Jun. 6, 2025

(30) Foreign Application Priority Data
Dec. 9, 2022 (CN) 202211578445.9
Feb. 3, 2023 (CN) 202310054915.X

(51) Int. Cl. C07K 14/445; C12N 15/79; G01N 33/531; G01N 33/575

(52) U.S. Cl. CPC C07K 14/445; C12N 15/79; G01N 33/531; G01N 33/5758; G01N 2333/445; G01N 2400/40; G01N 2800/50

(57) ABSTRACT

Provided is a recombinant VAR2CSA protein, and a preparation method and use thereof. The provided recombinant VAR2CSA protein and the preparation method thereof feature high stability and high yield, as well as good efficiency and capacity in detecting oncofetal chondroitin sulfate (ofCS) and an oncofetal chondroitin sulfate proteoglycan (ofCSPG), can be used for large-scale production of proteins for detection of ofCS/ofCSPG, and are used for early screening, diagnosis, tumor burden monitoring, and prognosis prediction of malignant tumors.

The present disclosure claims the benefits of Chinese Patent Application No. 202211578445.9 filed on Dec. 9, 2022 and Chinese Patent Application No. CN202310054915.X filed on Feb. 3, 2023, the contents of which are hereby incorporated by reference in their entireties.

### FIELD

The present disclosure belongs to the field of biomedicines, and relates to a recombinant VAR2CSA protein, and a preparation method and use thereof.

### BACKGROUND

A unique structural modification of chondroitin sulfate A (CSA) is commonly present in malignant tumor cells. The structure is a sugar chain composed of alternating disaccharide units form by glucuronic acid and N-acetylgalactosamine, with varying degrees of sulfate modification. This modified form is referred to as oncofetal chondroitin sulfate (ofCS) or placental chondroitin sulfate (plCS)¹. Due to its structural similarity to chondroitin sulfate found on the surface of placental trophoblast cells, ofCS can bind with high affinity to the VAR2CSA protein expressed by Plasmodium falciparum². The use of VAR2CSA-based ofCS detection technology holds significant potential for screening and diagnosis of malignant tumors.

Currently, the VAR2CSA protein has been employed in development of drugs targeting diseases with abnormal expression of chondroitin sulfate glycans, isolation of circulating tumor cells³ from the blood of tumor patients, and detection of ofCS-modified proteoglycans in the urine of bladder cancer patients⁴. Previous studies have expressed a VAR2CSA short peptide containing 28 amino acids, to construct an enzyme-linked immunosorbent assay (ELISA) detection system⁵ with VAR2CSA as a capture molecule and anti-ofCS antibodies as a detection molecule (Grant Publication No. CN 109387627 B). However, the affinity of this VAR2CSA short peptide for ofCS remains unclear, and the system suffers from limitations including low detection sensitivity (particularly in early-stage tumor detection), and the requirement for purified anti-ofCS antibodies. In another related disclosure (Application Publication No. CN 113740521 A), recombinant VAR2CSA (rVAR2) is used to detect a level of free ofCS in the urine of renal and bladder cancer patients, to diagnose cancer. Nevertheless, the ELISA method employed in the related disclosure is direct ELISA method, which also presents limitations such as unknown affinity of rVAR2 for CSA and insufficient sensitivity.

VAR2CSA is a large, multi-domain transmembrane protein (350 kDa) expressed on the surface of red blood cells infected with P. falciparum. Its extracellular domain includes an N-terminal segment (NTS), six Duffy-binding-like (DBL) domains, and three interdomain (ID) segments⁶. Existing studies have shown that the extracellular segment of VAR2CSA contains different CSA-binding domains, each with varying affinities⁷⁻⁹. A minimal CSA-binding domain of VAR2CSA has been identified as the DBL2X domain and its flanking interdomain^{10,11}. The full-length extracellular segment of VAR2CSA ensures high-affinity and specific binding to CSA¹². However, in vitro expression of a large recombinant protein of such the full-length extracellular segment presents technical challenges, including low yield and a high tendency for misfolding and instability of protein.¹³ Furthermore, high variability and genetic diversity of the var2csa gene may affect a CSA-binding effect. The large size and complex structure of the VAR2CSA protein, along with genetic variability across different isolates, complicate current strategies for large-scale production.¹⁴ Therefore, the key to developing VAR2CSA-based tumor biomarkers lies in stable production of small VAR2CSA fragments that retain high-efficiency detection capability for ofCS.

### SUMMARY

To address the issues of unstable expression and low yield associated with the full-length extracellular segment of the VAR2CSA protein currently available for ofCS detection, the present disclosure constructs VAR2CSA proteins that contain a minimal chondroitin sulfate A (CSA)-binding domain (ID1-DBL2X-ID2a) with varying sequence lengths and that are derived from different strains of Plasmodium falciparum. The present disclosure can be used for large-scale production of proteins for detection ofCS/an oncofetal chondroitin sulfate proteoglycan (ofCSPG), and are used for early screening, diagnosis, tumor burden monitoring, and prognosis prediction of malignant tumors.

In an aspect, the present disclosure provides a recombinant VAR2CSA protein, wherein the recombinant VAR2CSA protein comprises an amino acid having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as shown in any one of SEQ ID NO: 1 to SEQ ID NO: 4. In some embodiments, the recombinant VAR2CSA protein comprises an amino acid having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as shown in SEQ ID NO: 3. In some embodiments, the recombinant VAR2CSA protein is an isolated protein.

In an aspect, the present disclosure provides a nucleic acid molecule, wherein the nucleic acid molecule encodes the recombinant VAR2CSA protein. In some embodiments, the nucleotide acid molecule is selected from a nucleotide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleotide sequence as shown in any one of SEQ ID NO: 5 to SEQ ID NO: 8. In some embodiments, the nucleic acid molecule is selected from a nucleotide having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the nucleotide sequence as shown in SEQ ID NO: 7. In some embodiments, the nucleic acid is an isolated nucleotide.

In an aspect, the present disclosure provides a preparation method of a recombinant VAR2CSA protein, wherein the method comprises the following steps:
S1: constructing a nucleic acid molecule into an expression vector by homologous recombination using an amino acid sequence of the P. falciparum VAR2CSA protein;
S2: transforming a sequencing-verified positive clone that is prepared in step S1 into expression-competent cells;
S3: after screening, picking a single clone for a polymerase chain reaction (PCR) test; and
S4: adding an inducer to induce expression of a recombinant protein.

In some embodiments, a strain of P. falciparum is a P. falciparum strain FCR3 or 3D7; in some embodiments the number of the strain FCR3 is GenBank No: ADG23053.1; in some embodiments, the number of the strain 3D7 is NCBI: XP_001350415.1; in some embodiments, the expression vector is selected from a pGEX plasmid; in some embodiments, the pGEX plasmid is pGEX-4T2; in some embodiments, fusion expression of a protease recognition site and a tag protein is introduced at the C-terminus of the pGEX-4T2 plasmid; and
in some embodiments, the expression-competent cells are selected from Escherichia coli cells; in some embodiments, the screening in S3 is dual-antibiotic screening; in some embodiments, the dual antibiotics are ampicillin and/or streptomycin; and in some embodiments, the inducer is selected from isopropyl β-d-1-thiogalactopyranoside.

In some embodiments, the method further comprises: purifying the recombinant protein.

In some embodiments, the purifying the recombinant protein includes the following steps:
1) resuspending the expression-competent cells expressing the recombinant VAR2CSA protein to obtain a bacterial suspension, and lysing the bacterial suspension;
2) centrifuging the lysate;
3) filtering an upper supernatant obtained after centrifugation, adding to a chromatography medium that is equilibrated with a resuspension buffer and that is capable of binding to the C-terminal tag protein for fully incubation;
4) washing with a washing buffer, and eluting the recombinant VAR2CSA protein with an elution buffer;
5) loading the eluted protein product onto a glutathione S-transferase (GST) affinity chromatography medium that is equilibrated with a buffer for fully binding;
6) thoroughly washing with a buffer, replacing with a protease digestion buffer, and digesting overnight; and
7) passing a collected digestion product through a chromatography column capable of binding to the C-terminal tag protein again, collecting a protein eluate, performing buffer exchange by using a desalting column or PBS dialysis, and finally performing protein concentration.

In some embodiments, in step 1), the expression-competent cells are resuspended in a resuspension solution; in some embodiments, the resuspension solution includes 10 mM Na₂HPO₄, 1.8 mM KH₂PO₄ (pH=7.4), 140 mM NaCl, 2.7 mM KCl, 2.5 mM β-ME, 1 μM DNase I, 1 mM phenylmethylsulfonyl fluoride (PMSF), and a protease inhibitor I; in some embodiments, the resuspension solution is added at a ratio of the expression-competent cells to the resuspension solution being approximately 1 g:5 mL; in some embodiments, in step 1), bacterial suspension is lysed mechanically; in some embodiments, the bacterial suspension is lysed mechanically under the following conditions: the pressure is approximately 800, 900, 1000, 1100, or 1200 bar, and the bacterial suspension is lysed for 2 to 5 consecutive passes; in some embodiments, in step 2), the lysate is centrifuged at approximately 30,000× g, 40,000× g, or 50,000× g and approximately 3° C. to 5° C. for approximately 0.5, 1, 1.5, or 2 h; in some embodiments, in step 3), the upper supernatant obtained after centrifugation is filtered with a 0.45 μm and/or 0.22 μm filtration membrane; in some embodiments, in step 4), the washing buffer is a washing buffer with an imidazole concentration of approximately 80 to 150 mM; in some embodiments, in step 4), the elution buffer is an elution buffer with an imidazole concentration of approximately 300 to 700 mM; in some embodiments, in step 6), the digestion is performed at approximately 3° C., 4° C., or 5° C.; and in some embodiments, step 3) to step 7) are performed at approximately 2° C., 3° C., 4° C., 5° C., 6° C., 7° C., or 8° C.

In an aspect, the present disclosure provides an expression vector, wherein the expression vector comprises the nucleic acid; in some embodiments, the expression vector is selected from one or more of a group consisting of a plasmid, a bacteriophage, an artificial chromosome and a virus; in some embodiments, the expression vector is selected from a plasmid; in some embodiments, the plasmid is selected from a pGEX plasmid; and in some embodiments, the plasmid is selected from pGEX-4T2.

In an aspect, the present disclosure provides a cell, wherein the cell comprises the expression vector; in some embodiments, the cell is selected from a prokaryote and/or a eukaryote; in some embodiments, the prokaryote is selected from E. coli; and in some embodiments, E. coli is Shuffle T7 E. coli.

In an aspect, the present disclosure provides an enzyme-linked immunosorbent assay (ELISA) reagent for ofCS and/or an ofCS-modified proteoglycan, wherein the ELISA reagent comprises the recombinant VAR2CSA protein as a detection reagent; in some embodiments, the ELISA further comprises a capture reagent, the capture reagent is an antibody against ofCS and/or the ofCS-modified proteoglycan; in some embodiments, the antibody is a monoclonal antibody, a polyclonal antibody, a multispecific antibody, or an antibody fragment; in some embodiments, the capture reagent is a recombinant VAR2CSA protein; in some embodiments, the detection reagent further includes an enzyme labeling reagent; in some embodiments, the enzyme labeling reagent is horseradish peroxidase, alkaline phosphatase (ALP), β-galactosidase, or colloidal gold; in some embodiments, in a case of horseradish peroxidase, a chromogenic substrate is selected from 3,3′,5,5′-tetramethylbenzidine and o-phenylenediamine; in a case of ALP, a chromogenic substrate is selected from para-nitrophenyl phosphate; and in a case of β-galactosidase, a chromogenic substrate is selected from o-nitrophenyl-β-D-galactopyranoside; in some embodiments, the reagent further includes a blocking solution, a washing solution, a sample diluent, a colorimetric solution, a stop solution, and a standard. In some embodiments, the blocking solution is approximately 3% to 5% bovine serum albumin (BSA) or approximately 1% to 5% gelatin; and in some embodiments, the blocking solution is approximately 5% BSA.

In an aspect, the present disclosure provides use of the recombinant VAR2CSA protein, or the ELISA reagent in preparation of a detection reagent for ofCS and/or an ofCS-modified proteoglycan in a sample; in some embodiments, ofCS includes an ofCS glycosaminoglycan; in some embodiments, the ofCS-modified proteoglycan is selected from one or more of a group consisting of ofCS-modified CD44, ofCS-modified CSPG4, and ofCS-modified SDC1; and in some embodiments, the ofCS-modified proteoglycan is selected from ofCS-modified CD44.

In an aspect, the present disclosure provides use of the recombinant VAR2CSA protein, or the ELISA reagent in preparation of a detection reagent for detecting a tumor risk.

In some embodiments, the tumor is a CSA-expressing tumor; in some embodiments, the tumor is a malignant tumors of epithelial origin, a malignant tumor of mesenchymal origin, hematologic cancer, malignant melanoma, a malignant tumor of neuroepithelial tissue, or neuroendocrine carcinoma; in some embodiments, the malignant tumors of epithelial origin is breast cancer, pancreatic cancer, ovarian cancer, endometrial cancer, hepatocellular carcinoma, lung cancer, colorectal cancer, prostate cancer, cervical cancer, testicular cancer, basal cell carcinoma of the skin, clear cell renal cell carcinoma, keratinizing squamous cell carcinoma of the head and neck, squamous cell carcinoma of the skin, keratinizing squamous cell carcinoma of the vulva, basal cell carcinoma of the vulva, stomach cancer, thyroid cancer, intrahepatic cholangiocarcinoma, oral cancer, nasopharyngeal carcinoma, esophageal cancer, or bladder cancer; in some embodiments, the malignant tumor of mesenchymal origin is liposarcoma, fibrosarcoma, leiomyosarcoma, rhabdomyosarcoma, lymphangiosarcoma, or chondrosarcoma; in some embodiments, the hematologic cancer is lymphoma or leukemia; and in some embodiments, the malignant tumor of neuroepithelial tissue is glioma, diffuse astrocytoma, or neuroblastoma.

In an aspect, the present disclosure provides a method for detecting ofCS and/or an ofCS-modified proteoglycan, wherein the method comprises: contacting a sample with the recombinant VAR2CSA protein, or the ELISA reagent.

In some embodiments, the sample is selected from a group consisting of a body fluid and a tissue. In some embodiments, the body fluid is selected from one or more of a group consisting of plasma, serum, saliva, urine, cerebrospinal fluid, ascitic fluid, pleural effusion, and lavage fluid.

In an aspect, the present disclosure provides a method for detecting a tumor risk, wherein the method comprises:
(1) detecting a level of ofCS and/or an ofCS-modified proteoglycan in a sample obtained from a subject;
(2) comparing the level of ofCS and/or the ofCS-modified proteoglycan in the sample from the subject with that in a normal control sample; and
(3) determining whether the subject has a tumor or is at risk of developing a tumor based on a deviation of the level of ofCS and/or the ofCS-modified proteoglycan in the sample from the subject relative to that in the normal control sample, to distinguish a tumor sample from the normal sample.

In some embodiments, the level of ofCS and/or the ofCS-modified proteoglycan is detected using the recombinant VAR2CSA protein, or the ELISA reagent.

In an aspect, the present disclosure provides tumor detection system, wherein the system comprises the following components:
(1) a detection component for a level of ofCS and/or an ofCS-modified proteoglycan;
(2) a data processing component; and
(3) a result output component.

In some embodiments, the detection component for the level ofCS and/or the ofCS-modified proteoglycan further contains the recombinant VAR2CSA protein, or the ELISA reagent.

In some embodiments, the data processing component is configured to: a. receive test data of a to-be-tested sample from a subject and a normal control sample; b. store the test data of the to-be-tested sample from the subject and the normal control sample; c. compare the test data of the to-be-tested sample from the subject and the normal control sample of a same type; and d. determine, based on a comparison result, a probability or likelihood that the subject has a tumor.

In some embodiments, the result output component is configured to output the probability or likelihood that the subject has a tumor.

In some embodiments, a determination criterion of the data processing component is that: whether a sample is a tumor sample or a normal sample is determined based on a threshold.

In some embodiment of the present disclosure, four recombinant VAR2CSA sequences containing a minimal CSA-binding domain are constructed.

In some embodiments of the present disclosure, an expression vector pGEX-4T2 plasmid is modified to enhance solubility and yield of the protein, resulting in the successful expression of the VAR2CSA protein with significantly improved yield.

Among the four VAR2CSA sequences, the rVAR2-1 protein containing the most domain segments (DBL1X-ID1-DBL2X-ID2a-ID2b) exhibits the lowest yield, the rVAR2-4 protein containing domain segments ID1-DBL2X-ID2a-ID2b exhibit slightly higher yield, whereas the rVAR2-2 and rVAR2-3 proteins both containing the minimal domain segments (ID1-DBL2X-ID2a) exhibit relatively higher yield. Between rVAR2-2 and rVAR2-3, the yield of rVAR2-3 derived from the strain 3D7 is approximately twice the yield of rVAR2-2 derived from the strain FCR3.

The purified protein is subsequently labeled with horseradish peroxidase (HRP) and applied in ELISA. In a case-control cohort, a level of ofCS-CD44 is significantly elevated in a case group compared to a control group (p<0.0001). The area under the receiver operating characteristic (ROC) curve exceeds 0.8, demonstrating the effectiveness of this protein for malignancy detection (see FIG. 5).

In the present disclosure, the abbreviation “CSA” refers to chondroitin sulfate A, and the terms “CSA”, “chondroitin sulfate A”, and similar expressions are used interchangeably herein.

In the present disclosure, the abbreviation “rVAR2” refers to recombinant VAR2CSA, and the terms “rVAR2”, “recombinant VAR2”, “recombinant VAR2CSA”, and similar expressions are used interchangeably herein.

In the present disclosure, the abbreviation “ofCSPG” refers to oncofetal chondroitin sulfate proteoglycan. As glycosaminoglycans of ofCS are covalently attached to a plurality of proteins, different types of ofCSPGs exist depending on specific proteins to which ofCS binds, including but not limited to ofCS-CD44, ofCS-CSPG4, and ofCS-SDC1.

In the present disclosure, the term “detection” is the same as diagnosis. In addition to early diagnosis of cancer, it also includes diagnosis of mid- and late-stage cancer, and also includes cancer screening, risk assessment, prognosis, disease identification, diagnosis of disease stages, and selection of therapeutic targets.

The terms “about” or “approximately” refer to a deviation of within 10%, and more preferably within 5%, of the specified value or range.

### BRIEF DESCRIPTION OF THE DRAWINGS

FIG. 1 is a diagram of sodium dodecyl sulfate-polyacrylamide gel electrophoresis (SDS-PAGE) Coomassie blue staining results (A) and Western blot analysis results (B) of rVAR2-1 to rVAR2-4;

FIG. 2 is a diagram of summary of the mean fluorescence intensity of rVAR2-1 to rVAR2-4 binding to tumor cells;

FIG. 3 is a diagram of flow cytometry results of rVAR2-1 to rVAR2-4 binding to peripheral blood cells (ALL-P1: leukocytes from a patient with acute lymphoblastic leukemia—sample 1; and ALL-P2: leukocytes from a patient with acute lymphoblastic leukemia—sample 2);

FIG. 4 is a diagram of a principle of an enzyme-linked immunosorbent assay (ELISA) method according to the present disclosure; and

FIG. 5 is a diagram of the performance of rVAR2-1 to rVAR2-4 in detecting oncofetal chondroitin sulfate (ofCS)-CD44 in plasma from a case-control cohort (59 cases and 22 controls) in Example 4, and receiver operating characteristic (ROC) analysis of efficiency of ofCS-CD44 in detecting malignant tumors in the case-control cohort.

### DETAILED DESCRIPTION

The following further describes the technical solutions of the present invention with reference to specific examples, but the specific examples are not intended to limit the scope of protection of the present invention. Some nonessential modifications and adjustments made by others according to the idea of the present invention shall fall within the scope of protection of the present invention.

### Preparation Example

Cloning and Expression of Recombinant Vector in Escherichia coli

Fusion expression of a PreScission protease recognition site, EcoRI and HindIII restriction sites, a V5-tag, a TEV protease recognition site, and a His10-tag was introduced at the C-terminus of the BamHI restriction site in an original plasmid. Based on amino acid sequences of the Plasmodium falciparum VAR2CSAproteins (GenBank No: ADG23053.1) from the strain FCR3 and the strain 3D7 (NCBI: XP_001350415.1), a codon of a peptide segment was optimized with reference to the codon bias of E. coli. The target gene was then constructed into the expression vector by homologous recombination. For homologous recombination primers, see Table 1.

TABLE 1
Recombination primers for expression of rVAR2 insert in E. coli

| | | | Target gene |
|---|---|---|---|
| Protein | Sequence | Primer | sequence |
| rVAR2-1 | S-FCR3-F | TTCCAGGGGCCCGGTGAATTCAGCGATAGTGGCA | SEQ ID NO: 5 |
| | | AGTACG | |
| | S-FCR3-R | GATCGGCTTGCCACCAAGCTTCTGGCAACCACGG | |
| | | ATGTAGTT | |
| rVAR2-2 | S-FCR3-F | TTCCAGGGGCCCGGTGAATTCCAAGCGCAAACCA | SEQ ID NO: 6 |
| | | GCGGT | |
| | S-FCR3-R | GATCGGCTTGCCACCAAGCTTGTCCAGCTTGCTG | |
| | | C | |
| | | TGTTGC | |
| TVAR2-3 | S′-3D7-F | TTCCAGGGGCCCGGTGAATTCCAGGAGCAAATCA | SEQ ID NO: 7 |
| | | GCGACCC | |
| | S′-3D7-R | GATCGGCTTGCCACCAAGCTTCTGGCAACCGCGG | |
| | | ATGTA | |
| rVAR2-4 | S-FCR3-F | TTCCAGGGGCCCGGTGAATTCCAAGCGCAAACCA | SEQ ID NO: 8 |
| | | GCGGT | |
| | S-FCR3-R | GATCGGCTTGCCACCAAGCTTCTGGCAACCACGG | |
| | | ATGTAGTT | |

Sequencing-verified positive clones were transformed into Shuffle T7 E. coli competent cells. Following dual antibiotic screening with ampicillin and streptomycin, single clones were picked and subjected to a polymerase chain reaction (PCR) test again. The positive clones were inoculated into a lysogeny broth (LB) liquid medium containing ampicillin and streptomycin and thawed on a shaker at 37° C. for 12 h. The activated strain was then inoculated at a 1:100 ratio into a 2 L Erlenmeyer flask and further expanded on a shaker at 37° C. for 2 to 3 h. The shaker temperature was then adjusted to 18° C., and after the temperature stabilized, isopropyl β-D-1-thiogalactopyranoside (IPTG) was added to induce expression of the recombinant protein.

Purification of VAR2CSA Expressed in Recombinant Vector in E. coli

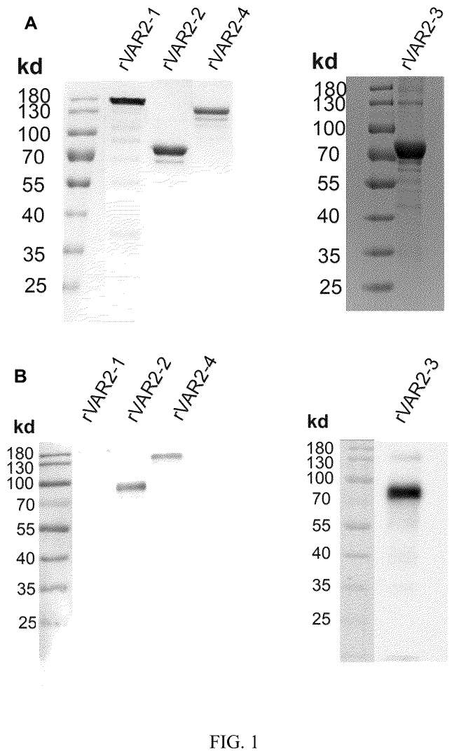

The bacterial cells were harvested and fully resuspended in a resuspension solution at a bacterial cell-to-resuspension solution ratio of 1 g:5 mL. The resuspension solution contained 10 mM Na₂HPO₄, 1.8 mM KH₂PO₄ (pH=7.4), 140 mM NaCl, 2.7 mM KCl, 2.5 mM β-ME, 1 μM DNase I, 1 mM phenylmethylsulfonyl fluoride (PMSF), and protease inhibitor I. The bacterial suspension was then mechanically lysed on ice at 1000 bar for three consecutive passes. The lysate was centrifuged at 40,000× g and 4° C. for 1 h. To prevent degradation of the recombinant protein, all subsequent operations were performed at 2° C. to 8° C. The upper supernatant obtained after centrifugation was filtered with 0.45 μm and 0.22 μm membranes, and the filtrate was loaded onto a Ni-NTA affinity column that was equilibrated with a resuspension solution. The column was washed thoroughly with a washing buffer containing 100 mM imidazole. After thorough washing, rVAR2 was eluted by using an elution buffer containing 500 mM imidazole. The eluted protein product was then incubated with glutathione S-transferase (GST) affinity chromatography medium that was equilibrated with phosphate-buffered saline (PBS). The mixture was rotated on a vertical rotator for 1 h to ensure sufficient binding of the recombinant protein to the medium. The protein-chromatography medium mixture was transferred into a gravity flow chromatography column, washed thoroughly with PBS after the liquid has flowed out, and then replaced with PreScission Protease digestion buffer. An appropriate amount of PreScission Protease was added, and the mixture was digested overnight at 4° C. On the next day, the digestion product was collected and passed through the Ni-NTA column again. The recombinant protein was eluted with an elution buffer containing 500 mM imidazole. The eluate was desalted using a desalting column and concentrated using an ultrafiltration tube. Protein concentration was determined by the bicinchoninic acid assay (BCA), and purity and identity were verified by sodium dodecyl sulfate-polyacrylamide gel electrophoresis (SDS-PAGE) and Western blot analysis (see FIG. 1).

TABLE 2
Information about four recombinant VAR2CSA proteins

| | | | Strain of P. | |
|---|---|---|---|---|
| Serial No. | Domains contained | Host cell | falciparum | Sequence No. |
| rVAR2-1 | DBL1X-ID1-DBL2X-ID2a-ID2b | E. coli | FCR3 | SEQ ID NO: 1 |
| rVAR2-2 | ID1-DBL2X-ID2a | E. coli | FCR3 | SEQ ID NO: 2 |
| rVAR2-3 | ID1-DBL2X-ID2a | E. coli | 3D7 | SEQ ID NO: 3 |
| rVAR2-4 | ID1-DBL2X-ID2a-ID2b | E. coli | FCR3 | SEQ ID NO: 4 |

### Example 1 Yield of VAR2CSA

After expression of VAR2CSA by using the recombinant vector in E. coli and subsequent purification, four recombinant VAR2CSA proteins were obtained. For yields of the four recombinant VAR2CSA proteins prepared in Preparation Example, see Table 3.

TABLE 3
Yields of four recombinant VAR2CSA proteins

| | Concen- | Bacterial | Total | | |
|---|---|---|---|---|---|
| | tration | culture | quantity | Yield | Strain of P. |
| Protein | (ug/uL) | volume (L) | (ug) | (ug/L) | falciparum |
| rVAR2-1 | 0.25 | 8 | 50 | 6.25 | FCR3 |
| rVAR2-2 | 0.58 | 2 | 116 | 58.1 | FCR3 |
| rVAR2-3 | 1 | 2 | 199 | 99.6 | 3D7 |
| rVAR2-4 | 0.44 | 4 | 87 | 21.9 | FCR3 |

Different VAR2CSA proteins exhibit variation in expression yields, with certain specific sequence structures being more conducive to stable expression, resulting in higher yields. The yield of rVAR2-2 reaches 58.1 μg/L, while rVAR2-3, having an equal sequence length, achieves an even higher yield of 99.6 μg/L. Such high yields are of significant importance in addressing the current challenges of unstable expression and low yields of the VAR2CSA proteins available for ofCS detection.

### Example 2 Flow Cytometry Detection of VAR2CSA Binding to Tumor Cells

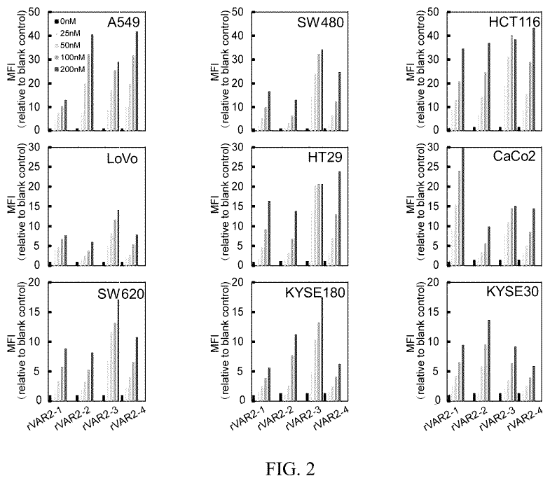

After being blocked, cells were incubated with rVAR2 for 1 h, washed and incubated with a fluorescein isothiocyanate (FITC)-labeled anti-V5 tag monoclonal antibody at room temperature in the dark for 1 h. After being washed again, the cells were resuspended in pre-cooled PBS solution containing 5% bovine serum albumin (BSA) and immediately analyzed using a flow cytometer. Fluorescence intensity was recorded for each group, and a ratio of the mean fluorescence intensity of each group to that of a blank control was taken as an indicator to evaluate the binding capacity of rVAR2 to the cells. Flow cytometry results demonstrate that all the four recombinant proteins can bind to lung adenocarcinoma cells (A549), colorectal cancer cells (SW480, HCT116, LoVo, HT29, CaCo2, and SW620), and esophageal squamous carcinoma cells (KYSE180 and KYSE30), and the mean fluorescence intensity increases with the increase of concentration of the incubated protein. (See FIG. 2).

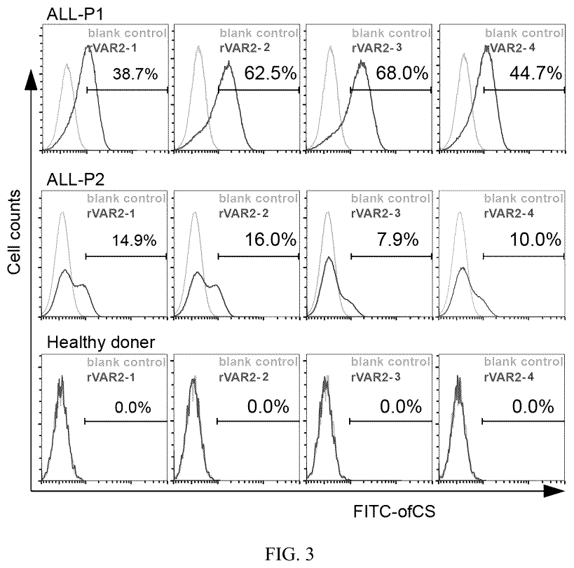

Additionally, rVAR2 can bind to peripheral blood leukocytes from patients with acute lymphoblastic leukemia, but does not bind to those from healthy controls (see FIG. 3).

This example confirms at the cellular level (see FIG. 2 and FIG. 3) that the prepared VAR2CSA proteins can specifically bind to ofCS and ofCSPG.

### Example 3 “Chessboard Method”—Optimized Sandwich ELISA Conditions

Determination of optimal coating concentration of antibody/rVAR2: coating concentration of an antibody and rVAR2 was set to 16 μg/mL, 8 μg/mL, 4 μg/mL, 2 μg/mL, 1 μg/mL, 0.5 μg/mL, 0.25 μg/mL, and 0.125 μg/mL, respectively. Determination of optimal coating buffer: candidate coating buffers included commonly used 0.05 M bicarbonate buffer (pH=9.6), 0.01 M Tris buffer (pH=8.0), and 0.01 M PBS buffer (pH=7.2). Determination of optimal blocking buffer: candidate blocking buffers included 1% gelatin, 3% gelatin, 5% BSA solution, and 5% milk. Determination of optimal plasma dilution: to-be-tested plasma samples were diluted across eight gradients ranging from 1:25 to 1:3200. Determination of optimal reaction time for to-be-tested samples: after to-be-tested plasma samples were added, reaction time was set to 30 min, 60 min, 90 min, and 120 min, respectively. Determination of optimal dilution of HRP-labeled rVAR2: HRP-labeled rVAR2 concentration was set to 3.2 μg/mL, 1.6 μg/mL, 0.8 μg/mL, 0.4 μg/mL, 0.2 μg/mL, 0.1 μg/mL, 0.05 μg/mL, and 0.025 μg/mL, respectively. Determination of optimal reaction time for HRP-labeled rVAR2: reaction time was set to 15 min, 30 min, 45 min, 60 min, 75 min, and 90 min, respectively. Determination of optimal 3,3′,5,5′-tetramethylbenzidine (TMB) reaction time: TMB reaction time was set to 5 min, 10 min, 15 min, 20 min, 25 min, and 30 min, respectively.

Each round of experiments followed a standardized operating procedure using both positive and negative plasma samples, with each sample tested in duplicate and the average value taken. The condition corresponding to the highest P/N value was defined as the optimal condition and used for subsequent steps. Finally, it is determined that the optimal reaction P/N value can be obtained by coating rVAR2-5, anti-CD44 monoclonal antibody, anti-SDC1 monoclonal antibody, and anti-CSPG4 polyclonal antibody at concentrations of 1 to 8 μg/mL, 1 to 5 μg/mL, 1 to 5 μg/mL, and 0.5 to 5 μg/mL, respectively.

A good reaction P/N value can be obtained by diluting the to-be-tested plasma at 1:5 to 1:100, and diluting HRP-labeled rVAR2-3 at a concentration of 0.1 μg/mL at 1:10 to 1:5000. Through time-gradient optimization, it is determined that the optimal reaction time for the to-be-tested plasma samples is 60 to 120 min, the HRP-labeled rVAR2-3 reaction time is 60 to 120 min, and the TMB development time is 5 to 30 min. In addition, a good reaction P/N value can be obtained by using 0.05 M bicarbonate buffer (pH=9.6) as the coating buffer and using 5% BSA solution as the blocking buffer.

### Example 4 Effect of VAR2CSA in Detecting ofCSPG in Case-Control Cohort

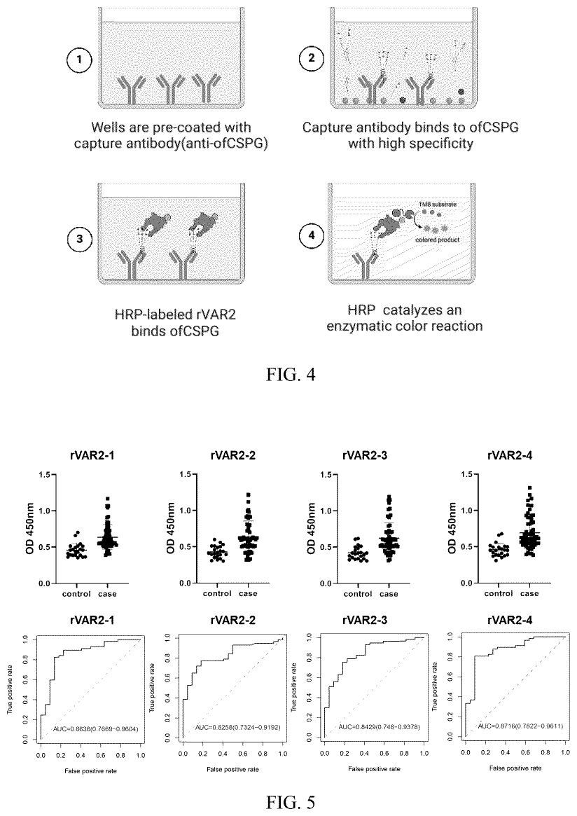

Detection principle: anti-CD44 antibodies were coated onto a 96-well ELISA plate and incubated overnight at 4° C. Unbound antibodies were washed off, and the wells were blocked with 5% BSA. Diluted to-be-tested plasma samples were added and incubated at room temperature, followed by incubation with HRP-labeled rVAR2 at room temperature. TMB was then added for color development (see FIG. 4).

Reaction parameters: the anti-CD44 antibody concentration was set to 1 to 8 μg/mL; the to-be-tested plasma sample was diluted at 1:5 to 1:100; HRP-labeled rVAR2 at a concentration of 0.1 μg/mL was diluted at 1:10 to 1:5000; the reaction time for the to-be-tested plasma samples was set to 60 to 120 min; the reaction time for HRP-labeled rVAR2 was set to 60 to 120 min; and the TMB development time was set to 5 to 30 min. A 0.05 M bicarbonate buffer (pH=9.6) was taken as a coating solution; and 5% BSA was taken as a blocking buffer.

A total of 22 healthy controls from the Guangdong natural population cohort (ChiCTR1800015736) and 59 tumor patients from the Sun Yat-sen University Cancer Center were included. Sandwich ELISA results demonstrate that the expression level of ofCS-modified CD44 in the plasma of the tested cancer patient is significantly higher than that in the healthy control (based on OD values at 450 nm). By taking ofCS-modified CD44 in plasma as an independent variable, logistic regression analysis was used to predict the probability of tumor occurrence. The areas under the curve (AUC) after age and gender adjustment are 0.864 (95% CI=0.7669 to 0.9604), Se=0.845, Sp=0.864; 0.8258 (95% CI=0.7324 to 0.9192), Se=0.793, Sp=0.727; 0.8429 (95% CI=0.748 to 0.9378), Se=0.879, Sp=0.682; and 0.8716 (95% CI=0.7822 to 0.9611), Se=0.810, Sp=0.864.

Se denotes sensitivity, Sp denotes specificity, and CI denotes confidence interval.
1. Salanti, A. et al. Targeting Human Cancer by a Glycosaminoglycan Binding Malaria Protein. Cancer Cell 28, 500-514 (2015).
2. Ma, R. et al. Structural basis for placental malaria mediated by Plasmodium falciparum VAR2CSA. Nat Microbiol 6, 380-391 (2021).
3. Agerbaek, M. O. et al. The VAR2CSA malaria protein efficiently retrieves circulating tumor cells in an EpCAM-independent manner. Nat Commun 9, 3279 (2018).
4. Clausen, T. M. et al. A simple method for detecting oncofetal chondroitin sulfate glycosaminoglycans in bladder cancer urine. Cell Death Discov 6, 65 (2020).
5. Zhang, J. et al. Screening and surveillance of multiple solid tumours using plasma placental-like chondroitin sulfate A (pl-CSA). Int J Med Sci 17, 161-169 (2020).
6. Jagadeeshaprasad, M. G. et al. Disulfide bond and crosslinking analyses reveal inter-domain interactions that contribute to the rigidity of placental malaria VAR2CSA structure and formation of CSA binding channel. Int J Biol Macromol 226, 143-158 (2023).
7. Bir, N. et al. Immunogenicity of Duffy binding-like domains that bind chondroitin sulfate A and protection against pregnancy-associated malaria. Infect Immun 74, 5955-63 (2006).
8. Gamain, B. et al. Identification of multiple chondroitin sulfate A (CSA)-binding domains in the var2CSA gene transcribed in CSA-binding parasites. J Infect Dis 191, 1010-3 (2005).
9. Resende, M. et al. Chondroitin sulphate A (CSA)-binding of single recombinant Duffy-binding-like domains is not restricted to Plasmodium falciparum Erythrocyte Membrane Protein 1 expressed by CSA-binding parasites. Int J Parasitol 39, 1195-204 (2009).
10. Sugiura, N. et al. Construction of a chondroitin sulfate library with defined structures and analysis of molecular interactions. J Biol Chem 287, 43390-400 (2012).
11. Dahlback, M. et al. The chondroitin sulfate A-binding site of the VAR2CSA protein involves multiple N-terminal domains. J Biol Chem 286, 15908-17 (2011).
12. Srivastava, A. et al. Full-length extracellular region of the var2CSA variant of PfEMP1 is required for specific, high-affinity binding to CSA. Proc Natl Acad Sci USA 107, 4884-9 (2010).
13. Ferrer-Miralles, N., Saccardo, P., Corchero, J. L., Xu, Z. & Garcia-Fruitos, E. General introduction: recombinant protein production and purification of insoluble proteins. Methods Mol Biol 1258, 1-24 (2015).
14. Clausen, T. M. et al. Structural and functional insight into how the Plasmodium falciparum VAR2CSA protein mediates binding to chondroitin sulfate A in placental malaria. J Biol Chem 287, 23332-45 (2012).

1-15. (canceled)

16. A recombinant VAR2CSA protein, wherein the recombinant VAR2CSA protein comprises an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS:1-4.

17. A nucleic acid molecule, wherein the nucleic acid molecule encodes the recombinant VAR2CSA protein according to claim 16.

18. The nucleic acid according to claim 17, wherein the nucleic acid molecule comprises a nucleotide sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS:5-8.

19. A preparation method of the recombinant VAR2CSA protein according to claim 16, wherein the method comprises the following steps:
i) constructing a nucleic acid molecule encoding the recombinant VAR2CSA protein according to claim 16 into an expression vector by homologous recombination using an amino acid sequence of a Plasmodium falciparum: VAR2CSA protein;
ii) transforming a sequencing-verified positive clone that is prepared in step i) into expression-competent cells;
iiii) after screening, picking a single clone for a polymerase chain reaction (PCR) test; and
iv) adding an inducer to induce expression of the recombinant protein.

20. The method according to claim 19, wherein the amino acid sequence of the P. falciparum VAR2CSA protein is from strain FCR3 or 3D7.

21. The method according to claim 19, wherein the method further comprises: purifying the recombinant protein, wherein purifying the recombinant protein comprising the following steps:
1) resuspending the expression-competent cells expressing the recombinant VAR2CSA protein to obtain a bacterial suspension, and lysing the bacterial suspension;
2) centrifuging the lysate;
3) filtering an upper supernatant obtained after centrifugation, and adding the filtered upper supernatant to a chromatography column that is equilibrated with a resuspension buffer and that is capable of binding to a C-terminal tag protein;
4) washing the chromatography column with a washing buffer, and eluting the recombinant VAR2CSA protein with an elution buffer;
5) loading the eluted recombinant VAR2CSA protein onto a glutathione S-transferase (GST) affinity chromatography medium that is equilibrated with a buffer;
6) thoroughly washing with a buffer, replacing with a protease digestion buffer, and digesting overnight; and
7) passing a collected digestion product through a chromatography column capable of binding to the C-terminal tag protein again, collecting a protein eluate, performing buffer exchange by using a desalting column or PBS dialysis, and finally performing protein concentration.

22. An expression vector, wherein the expression vector comprises the nucleic acid molecule according to claim 17.

23. A cell, wherein the cell comprises the expression vector according to claim 22.

24. An enzyme-linked immunosorbent assay (ELISA) reagent for oncofetal chondroitin sulfate (ofCS) and/or an ofCS-modified proteoglycan, wherein the ELISA reagent comprises the recombinant VAR2CSA protein according to claim 16 as a detection reagent.

25. The ELISA reagent according to claim 24, wherein the ELISA further comprises a capture reagent, and the capture reagent is a recombinant protein, and/or
an antibody against ofCS and/or the ofCS-modified proteoglycan.

26. A method for detecting ofCS and/or an ofCS-modified proteoglycan for detecting a tumor, wherein the method comprises: contacting a sample with the recombinant VAR2CSA protein according to claim 16.

27. The method according to claim 26, wherein the tumor is a CSA-expressing tumor.

28. The method according to claim 26, wherein the sample is selected from a group consisting of a body fluid and a tissue.

29. The method according to claim 28, wherein the body fluid is selected from one or more of a group consisting of plasma, serum, saliva, urine, cerebrospinal fluid, ascitic fluid, pleural effusion, and lavage fluid.

30. The method according to claim 26, wherein the tumor is a malignant tumor of epithelial origin, a malignant tumor of mesenchymal origin, hematologic cancer, malignant melanoma, a malignant tumor of neuroepithelial tissue, or neuroendocrine carcinoma.

31. A method for detecting a tumor risk, wherein the method comprises:
(1) detecting a level of ofCS and/or an ofCS-modified proteoglycan in a sample obtained from a subject;
(2) comparing the level of ofCS and/or the ofCS-modified proteoglycan in the sample from the subject with that in a normal control sample; and
(3) determining whether the subject has a tumor or is at risk of developing a tumor based on a deviation of the level of ofCS and/or the ofCS-modified proteoglycan in the sample from the subject relative to that in the normal control sample, to distinguish a tumor sample from the normal sample;
the level of ofCS and/or ofCS-modified proteoglycan being detected using the recombinant VAR2CSA protein according to claim 16.

32. The method according to claim 31, wherein the tumor is a CSA-expressing tumor.

33. The method according to claim 31, wherein the sample is selected from a group consisting of a body fluid and a tissue.

34. The method according to claim 33, wherein the body fluid is selected from one or more of a group consisting of plasma, serum, saliva, urine, cerebrospinal fluid, ascitic fluid, pleural effusion, and lavage fluid.

35. The method according to claim 31, wherein the tumor is a malignant tumor of epithelial origin, a malignant tumor of mesenchymal origin, hematologic cancer, malignant melanoma, a malignant tumor of neuroepithelial tissue, or neuroendocrine carcinoma.
